(19) United States
(12) Patent Application Publication
(10) Pub. No.: US 2010/0113746 A1
(43) Pub. Date: May 6, 2010

(54) PROCESS FOR PURIFICATION OF IMMUNOGLOBULINS USING A PSEUDOBIOAFFINITY ADSORBENT

(75) Inventors: Arvind Mallinath Lali, Mumbai (IN); Amith Dattatray Naik, Mumbai (IN); Monika Raina, Mumbai (IN); Sandeep Bhaskar Kale, Mumbai (IN)

Correspondence Address: LUCAS & MERCANTI, LLP, 475 PARK AVENUE SOUTH, 15TH FLOOR, NEW YORK, NY, 10016, US

(73) Assignee: Arvind Mallinath LALI, Mumbai (IN)

(21) Appl. No.: 12/597,136

(22) PCT Filed: Apr. 23, 2008
(86) PCT No.: PCT/IN2008/000254

§ 371 Date: Oct. 22, 2009

(30) Foreign Application Priority Data
Apr. 23, 2007 (IN) 635/MUM/2006

(51) Int. Cl. C07K 16/00; B32B 5/18

(52) U.S. Cl. 530/387.1; 428/319.3; 428/319.7

(57) ABSTRACT

The present invention relates to the development of pseudobioaffinity adsorbent and process for purification of immunoglobulin G from immunoglobulin containing solutions such as but not limited to plasma, serum, cell culture supernatant, ascites fluids. The adsorbent consists of a solid support and ligand. The ligand may be attached to the matrix or be part of matrix. The ligand is selected from a group of hydrophobic amino acids such as alanine, valine, leucine, isoleucine, proline, phenylalanine, tryptophan and tyrosine and the support material being preferably a synthetic hydrophilic polymer of methacrylate or acrylate species, or any of its derivatives. The adsorbent is cheap and stable to harsh conditions such as 1.0 M NaOH used during regeneration of adsorbents. Moreover there is no problem of toxic leachables typically associated with biological ligands. The nature of the adsorbent also allows high flow rate operations at relatively low pressures. The process includes adjusting the pH and conductivity of the feed solution and the use of ionic salts and additives such as polyols or alcohols for eluting the bound IgG.

### FIELD OF INVENTION

The present invention relates to a ‘novel pseudobioaffinity adsorbent’ and a process for purification of Immunoglobulin G (IgG) also known as antibodies, and fragments thereof such as Fab, Fc and F(ab)₂ from their solutions such as, but not limited to, plasma, serum, cell culture supernatant, ascites fluids, transgenics or dried/semidried or lyophilized crude material obtained from said sources, and through use of the designed ‘novel pseudobioaffinity adsorbent’.

### BACKGROUND OF THE INVENTION (PRIOR ART)

Immunoglobulins (Igs) or antibodies are proteins produced by the immune system to identify and neutralize foreign objects. In mammalians these are subdivided into five distinct subclasses viz. IgA, IgD, IgE, IgG and IgM. Immunoglobulin G (IgG) is the major class accounting for about 75% of the total immunoglobulins. The average concentration of IgG in adult human blood is about 12 mg/ml. Human IgG, in turn, is composed of four subclasses in approximate proportions as: 56% IgG1, 34% IgG2, 6% IgG3 and 4% IgG4 (Hahn et al., J. Chromatogr. B., 2003, 790:35-51). The IgGs present in the mammalian blood are produced by different types of immune cells and hence are called as polyclonal IgG or polyclonal antibodies. Monoclonal antibodies are antibodies that are produced by only one type of immune cell.

Both polyclonal and monoclonal antibodies have a plethora of applications in modern biotechnology as in areas such as immunodiagnostics, epitope mapping and therapeutics. Polyclonal antibodies, or Immunoglobulin G (IgG), preparations isolated from human plasma, or hyperimmune plasma, or sera, have been used in treatment of a number of congenital and acquired immunodeficiency diseases and infectious diseases. Monoclonal antibodies, on the other hand, constitute today one of the major and fastest growing classes of biopharmaceuticals used for indications such as transplant rejection, cancer, arthritis etc.

Traditionally, polyclonal IgG is produced from mammalian blood serum by a method called Cohn plasma fractionation and is based on differential precipitation of proteins using cold ethanol (Cohn et al., J. Am. Chem. Soc., 1946, 68: 459-475). IgG isolated by this method tends to aggregate to form multimers (J. Harris, Blood Separation and Plasma Fractionation, John Wiley & Sons, New York, 1990, 325p). Further, Cohn fractionation suffers from limitations such as inability to inactivate blood-carried viruses, poor product yield, difficulty in automation and loss of product due to use of harsh conditions and chemicals such as ethanol which are known to affect the biological function of polyclonal IgG. Other precipitation methods using inorganic salts such as ammonium sulphate, potassium phosphate, organic polymers such as polyethylene glycol, organic acids such as octanoic acid (Steinbuch et al., Rev. Franc. Et. Clin. et Biol., 1969, XIV, 1054; US Patent publication No. 2004132979A1), and organic bases such as ethacridine have been reported for isolation of IgG. Wo/2006/064373 discloses the method of purifying immunologlobulins by precipitating albumin from a biological fluid or partially purified fraction with caprylic acid, thereby yielding a first precipitate and then precipitating euglobulin Ig by desalting.

However, all precipitation methods, (using ethanol, polyethylene glycol, lyotropic; anti-chaotropic, ammonium sulfate and potassium phosphate, and caprylic acid) besides being tedious and less elegant, suffer from limitations such as low purity and low recovery of the final product. Furthermore, the addition of the precipitating agent to the raw material makes it difficult to use the supernatant for other purposes and creates a disposal problem. This is particularly relevant when speaking of large scale purification of immunoglobulins.

WO/1999/064462 discloses the combination of precipitation, anion exchange, cation exchange and filtration, for purification of immunoglobulins. The product of the invention is more than 95% pure. WO/1998/005686 discloses the enhancement of purification of immunoglobulins by using macro-porous ion exchange resins. WO/2002/092632 discloses method for preparing human immunoglobulin concentrates for therapeutic use, from plasma or a plasma fraction by pre-purification and a single anion-exchange chromatography carried out at alkaline pH, thereby enabling the immunoglobulins to be retained on the chromatographic support. The method enables to obtain IgG, IgA and IgM concentrates. EP 0703922 and WO 99/64462 discloses two successive chromatography steps, one using anion exchange and the other using cation exchange for preparing immunoglobulin concentrates. WO/1995/022389 discloses the use of hydrophobic interaction chromatography for purification of immunoglobulins at more than 95% purity. US 2003/0229212 discloses the use of ion exchange chromatography (IEC) specifically cation exchange, and hydrophobic charge chromatography (HIC) for purification of antibodies from mixture containing host cell proteins. Hydrophobic charge induction chromatography (HCIC) has also been reported for IgG purification, and gave a product purity of 98% when the feedstock solution was protein-free cell culture supernatant (Guerrier, et al., J. Chromatogr. B, 2001, 755(1-2): 37-46; Guerrier, et al., Bioseparation, 2000, 9(4): 211-221.

CA2604877A1 discloses the combination of the two methods i.e. ion exchange chromatography (IEC) and hydrophobic interaction chromatography (HIC) for purification of IgG from cell culture supernatant containing 5% FCS. The IEC step was coupled to hydrophobic charge induction chromatography (HCIC), and the purity and recovery of IgG were found to be 69% and 76%, respectively. A combination of methods, i.e. a caprylic acid precipitation and ion exchange chromatography, was used by Raweerith, R. et al. (J. Immun. Meth. 282 (2003) 63-72), as means to fractionate pepsin-digested horse antivenom F(ab′)₂ antibody. Necina, R. et al., (Biotechnol. Bioeng. 60 (1998) 689-698), reported the capture of human monoclonal antibodies directly from cell culture supernatants by ion exchange media exhibiting high charge density. The process gave a recovery of less than 70%.

In WO 89/05157 a method is reported for the purification of product immunoglobulins by directly subjecting the cell culture medium to a cation exchange treatment. A one-step purification of monoclonal IgG antibodies from mouse ascites is described by Danielsson, A., et al., (J. Immun. Meth. 115 (1988), 79-88). Follman, D. K., and Fahrner, R. L., reported a factorial screening of antibody purification processes using three chromatography steps without protein A (J. Chrom. A 1024 (2004) 79-85). Mhatre, R. et al. (J. Chrom. A 707 (1995) 225-231), explored the purification of antibody Fab fragments by cation exchange chromatography and pH gradient elution.

Ion exchange chromatography though reported for immunoglobulins, is not generally a preferred method because of the constraints it requires on ionic strength and pH necessary to ensure efficient binding of the antibodies since immunoglobulins display varying Isoelectric points of different immunoglobulins. The disadvantage of an HIC based process is the necessity to add lyotropic salt/s to the feed to result in effective binding. Use of salt on the one hand, presents disposal problems, and promotes aggregation of immunoglobulins as well. For some sources of antibodies other than cell culture supernatants such as whey, plasma, and egg yolk, addition of lyotropic salts to feed materials for HIC can in many instances be prohibitive in large scale applications as the added salt would prevent any further use of the immunoglobulin depleted raw material while also presenting disposal problem for several thousand liters of waste. Most important, techniques like IEC, HIC and HCIC suffer from low binding specificity and low yields, compared to more elegant affinity chromatographic methods.

Many immunoglobulin specific chromatography methods such as affinity chromatography using ligands such as Protein A from Staphylococcus aureus, Protein G from Streptococcus G, and more recently Protein L from Peptostreptococcus mugnus, have been reported. WO 2004/076485 reports a process for antibody purification by Protein A and ion exchange chromatography. Process yielded at least 90% of the amount of antibody loaded onto the ion exchange material in the flow-through of the ion exchanger whilst any contaminant Protein A or Protein A derivative is bound to the ion exchange material. In EP 0530447 a process for purifying IgG monoclonal antibodies by a combination of three chromatographic steps is reported. The removal of Protein A from antibody preparations is reported in U.S. Pat. No. 4,983,722.

WO 95/16037 reports the purification of anti-EGF-R/anti-CD3 bispecific monoclonal antibodies from hybrid hybridoma performed by combination of Protein A and cation exchange chromatography. The separation of antibody monomers from its multimers by use of ion exchange chromatography is reported in EP 1084136. U.S. Pat. No. 5,429,746 relates to the application of hydrophobic interaction chromatography and Protein A combination to the purification of antibody. WO/2006/125599 reports a process for antibody purification using ion exchange resins from Protein A eluate. The yields of as high as 95% are reported. PCT/SE83/00297 discloses a recombinant form of Protein A, wherein a cysteine residue has been added to the Protein A molecule to improve its coupling to a separation matrix for subsequent use as an affinity ligand for immunoglobulins. The combination of two affinity chromatography steps is reported in WO/92/19973. WO/2000/074728 discloses detection and purification of Fe and Fab fragments of IgG's using reagents prepared from the B1 domain of bacterial Protein G.

Despite popular use of these biological affinity ligands, it is recognized that Protein A and Protein G pose several problems to the user. These problems relate to leakage of Protein A/Protein G into the product, and low stability of the matrix in typical cleaning solutions, e g 1 M sodium hydroxide. Other problems are high cost and variable binding efficiency of different monoclonal antibodies (particularly mouse IgGi). Leakage of the protein based biological ligands like those mentioned above, necessitates further processing of the purified IgG preparations: On the other hand, leakage of the protein ligands also limits the life of the expensive affinity adsorbent. Another major drawback of the mentioned biological affinity adsorbents relates to in-place cleaning and sanitization problems since these biological protein based ligands arc fragile, and are easily damaged by presence of proteases in biological feeds, and cannot be subjected to extreme pH, chaotropes, detergents etc. used for cleaning of the adsorbents intended for production of therapeutic products. The proteases in the feed streams reduce the efficacy of the ligand is a protein, and the fragments generated due to the proteolytic action contaminate the antibody or IgG preparations. Yet another drawback of the protein (A, G or L) based biological ligands in use today is the use of low pH conditions required for elution of IgG from the affinity adsorbents. Low pH is known to not only affect the bioactivity of the product; it also leads to aggregation of IgG making further polishing of IgG more stringent. Aggregation of products also leads to low yields. Each of these drawbacks has its specific consequence in the individual application, ranging from insignificant to very serious and prohibitive consequences.

Many attempts have been made all over the world to overcome the above problems seen with the reported biological affinity ligands. A number of non-biological synthetic ligands have been reported and these ‘pseudo-affinity’ ligands are claimed to display equivalent, or near-equivalent affinity as biological ligands for IgG. These non-biological ligands, called, pseudobioaffinity or pseudoaffinity ligands are immobilized using suitable chemistry on suitable adsorbent matrices to result in pseudobioaffinity adsorbents. Thus, pseudobioaffinity adsorbents such as based on histidyl, thiophilic, organic chromophoric dye molecules and other low molecular weight molecules have been reported for purification of IgG.

El-Kak and Vijayalakshmi (J. Chromatogr. Biomed. Appl., 1991, 570: 29-41), has reported chromatographic purification of IgG using an amino acid histidine and histamine as pseudobioaffinity ligands for the purification of monoclonal antibodies. Histidine and histamine were coupled to Sepharose 4B, with and without a spacer arm. IgG1 was reported as not completely purified with histidyl Sepharose and the recovery was low, whereas the binding capacity of the histidyl-aminohexyl-Sepharose and histamine-Sepharose for both IgG1 and IgG2a was low. Wu et al. (J. Chromatogr. Biomed. Appl., 1992, 584: 35-41) also reported use of histidine which was coupled to a silica support. Haupt et al. (J. Chromatogr. B., 1995, 674:13-21) reported use of histidine that was coupled to poly(ethylene vinyl alcohol) hollow fiber filtration membrane for purification of IgG.

Thiophilic adsorption chromatography for purification of IgG was reported by Porath et al. (FEBS Letters, 1985, 185: 306-310). Porath and Belew (Trends Biotechnol., 1987, 5: 225-229) used a synthesized thiophilic gel to bind IgG in presence of high salt concentration. The bound IgG was eluted out by decreasing the salt concentration. Thiophilic gel used was prepared by reacting either a hydroxyl or thiol containing support, first with divinyl sulfone and then with 2-mercaptoethanol. U.S. Pat. No. 4,897,467 and Schwarz et al. (J. Mol. Recognit., 1996, 9: 672-674) also disclosed use of thiophilic compounds as ligands for purification of IgG. Nopper et al. (Anal. Biochem., 1989, 180: 66-71) developed a silica based thiophilic gel for purifying IgG. They prepared the affinity adsorbent by reacting epoxy-activated silica gel first with sodium hydrosulfide and divinyl sulfone, and then with 2-mercaptoethanol. All methods using thiophilic adsorbents utilized salt promoted adsorption of IgG, and elution of the bound IgG was performed by lowering the pH and salt concentration. It has been postulated that the sulfone group from the divinyl sulfone spacer and thio ether group in the ligand are responsible for the binding specificity of these affinity adsorbents for IgG.

However, use of thiophilic affinity adsorbents has not found acceptance and have not been able to replace the biological ligands like Protein A and Protein G because they also have a major disadvantage in that it is needed to add lyotropic salts to the raw material to ensure efficient binding of the immunoglobulin, which is a problem for the reasons discussed above.

Further attempts have been reported that try to overcome the specificity problems of the thiophilic ligands through use of mercaptoheterocylic based ligands (U.S. Pat. No. 6,610,630; U.S. Pat. No. 6,919,021). Ngo and Khatter (J. Chromatogr., 1990, 510: 281-291; J. Chromatogr., 1992, 597: 101-109) reported use of chromatographic methods employing pyridine based ligands for the purification of IgG. The synthetic affinity gel was prepared by first reacting hydroxyl groups of a support like Sepharose with pentafluoropyridine and 4-dimethylaminopyridine in an anhydrous polar organic solvent, and then reacting the gel with nucleophiles such as ethylene glycol or glycine in basic aqueous solutions.

Another class of pseudoaffinity ligands reported for the affinity purification of IgG is peptide based ligands. Fassina et al. (J. Mol. Recognit., 1996, 9: 564-569) reported use of a tetrameric peptide for the affinity purification of IgG. To increase the resistance to protease degradation the above designed tetrameric ligand was modified by replacing all the amino acids with their D-form (Verdolivia et al., J. Immunological Methods, 2002, 271: 77-88). Fassina et al. (Peptides 1994. Leiden: ESCOM. 1995, 489-490) reported a cyclic peptide based ligand for IgG purification. Li et al. (Nat. Biotechnol., 1998, 16: 190-195) developed a nonpeptidyl mimic for Staphylococcus aureus Protein A. The mimic consists of an aniline and tyramino groups attached to a triazine ring.

WO/2007/004954 discloses [1,2,4]triazolo[1,5-a]pyrimidine derivatives as chromatographic adsorbent for the selective adsorption of IgG. WO2004039765 describes the use of phenyl urea scaffold based small molecules as chromatography affinity ligands for IgG and Fab fragments with light chain of kappa-type. U.S. Pat. No. 6,610,630 describes the use of 2-mercaptoimidazole and derivatives thereof attached to a solid support as pseudo bio-affinity chromatography media for selective adsorption of IgG. U.S. Pat. No. 6,117,996 describes the preparation of triazine based structures and their use in the purification of various proteinaceaous materials. US 20030166002 describe the synthesis and selection of active compounds based on triazine structures carrying a linker suited for attachment to a resin. EP 1500431 relates to a medium, which comprises a solid support and, attached thereto, one or more affinity chromatographic ligands selected from 2-aminobenzimidazole and 2-aminomethylbenzimidazole. The affinity ligands of the invention are used for IgG purification.

WO 96/00735 and WO 96/09116 discloses matrix for purifying proteins and peptides characterized by the fact that they contain ionizable ligands and/or functionalities which are uncharged at the of binding the target protein or peptide, thereby facilitating hydrophobic interactions, and charged at the pH of desorption, thereby disrupting the established hydrophobic interaction between the resin and the target protein or peptides. WO 96/00735 mentions the possibility of coupling 2-mercapto-benzimidazole to epoxy-activated Sepharose 6 B. The actual ligand concentration is not disclosed; however the coupling was performed with an epoxy-activated. WO 92/16292 discloses a number of different ligands coupled to divinyl sulfone activated agarose and the use of the resulting solid phase matrices for thiophilic adsorption of proteins, preferably immunoglobulins. Specifically is mentioned solid phase matrices comprising 4-amino-benzoic acid as a ligand on a divinyl sulfone activated agarose The adsorption of proteins, preferably immunoglobulins in WO 92/16292, is performed at high concentrations of lyotropic salts. In WO 2003/102132 the combination of a non-affinity purification step and a high performance tangential-flow filtration is reported for the purification of proteins.

WO/1998/008603 discloses various ligands coupled to solid matrices with or without spacer arm, for isolation or purification of immunoglobulins from various raw materials. The patent does not disclose the purification of immunoglobulin fragments. The patent does not disclose the use of propanoic acid or derivatives of propanoic acid such as amino acids coupled to solid matrix as a pseudobioaffinity ligand for purification of immunoglobulins from raw materials. Patent also does not disclose the specificity of the ligands for any target protein. The Patent describes polyamides such as polyacrylamides and polymethacrylamides and poly (meth) acryl-amides and does not describe the use of methacrylate or polymethacrylate as a base matrix for conjugation or coupling of any ligands. Further, the patent discloses the use of organic solvent such as 1,2-propanediol, less than 10% for the elution, indicates that ligands are used as hydrophobic interaction chromatography. It is believed that use of organic solvents for elution from the ligands can have deleterious effect on bioactivity and stability of the immunoglobulins.

Although the processes of prior art employing low molecular weight and peptide based pseudoaffinity ligands have been able to address some of the problems like cost, physical and chemical stability of both the affinity adsorbent and the product IgG, there still remain one or the other problem issues such as leakage of ligands, toxicity of the ligands, non-specific protein binding (resulting in low purity) and low IgG binding capacity, yield and purity of IgG as compared to biological affinity based affinity adsorbents like those based on Protein A, G or L. Therefore, designing of novel pseudobioaffinity absorbent and the process for the purification of immunoglobulins and/or fragments thereof is highly desirable.

None of the prior art discloses the use of propionic acid or its derivatives as ligands such as in hydrophobic amino acid, which are non-toxic, for purification of immunoglobulins and/or fragments thereof with very high degree of specificity and selectivity from crude sources, and at high yield and purity of immunoglobulins. Therefore, it is the object of present invention to develop (a) novel inexpensive, physical and chemically stable, non-toxic, specific pseudobioaffinity adsorbent for IgG, and (b) the process for the purification of immunoglobulin G (IgG) using the developed adsorbent.

### BRIEF DESCRIPTION OF THE PRESENT INVENTION

The primary objective of this invention is to develop a pseudobioaffinity adsorbent and a process for purification of immunoglobulin G (IgG) and fragments thereof viz. Fab, Fc and F(ab)₂ using the said adsorbent. Developed affinity adsorbent having a high selectivity for IgG and fragments thereof comprises of a) solid support material and b) a ligand immobilized on the support material, the ligand being a hydrophobic amino acid, and the support material being preferably a synthetic hydrophilic polymer of methacrylate or acrylate species or any of its derivatives.

The developed process comprises of:
1. Adjusting the pH and conductivity of the immunoglobulin, and/or fragments thereof, containing solution,
2. Contacting the solution obtained from step 1 or a crude source directly with the said adsorbent (may or may not be pre-equilibrated),
3. Washing the said adsorbent (optional),
4. Contacting the said adsorbent with an eluent buffer solution in order to desorb/elute the bound immunoglobulin and/or fragments thereof in purified form, and
5. Regenerating and equilibrating the said adsorbent for reuse

Additional features and advantages of the present invention will be set forth in part in the description that follows, and in part will be apparent from the examples, or may be learned by practice of the present invention. The objectives and other advantages of the present invention will be realized and attained by means of the elements and combinations particularly pointed out in the description and appended claims. It is to be understood that both the foregoing general description and the following examples are exemplary and explanatory only and are intended to provide further explanation of the present invention, as claimed.

### BRIEF DESCRIPTION OF THE DRAWINGS

FIG. 1a: SDS PAGE analysis (non-reducing) of chromatographic runs of diafiltered human plasma on pseudobioaffinity adsorbent. Lane 1: Molecular weight marker, Lane 2: Load (Human plasma), Lane 3: Unbound fraction, Lane 4: Elution fraction, Lane 5: Molecular weight marker.

FIG. 1b: SDS PAGE analysis (non-reducing) of chromatographic runs of diafiltered horse plasma on pseudobioaffinity adsorbent. Lane 1: Molecular weight marker, Lane 2: Unbound fraction, Lane 3: Load (Horse plasma), Lane 4: Elution fraction, Lane 5: Molecular weight marker.

FIG. 2a: Chromatogram showing the single peak (2) of IgG as elution and other protein in the flow through fraction.

FIG. 2b: Adsorption isotherm for immunoglobulin G on said pseudobioaffinity adsorbent.

FIGS. 3a and 3b: HPLC chromatograms on SEC Biosil-250 column (BioRad, USA) for standard immunoglobulin and elution fraction (purified IgG) using said pseudobioaffinity adsorbent.

### DETAILED DESCRIPTION OF INVENTION

The present invention relates to development of pseudobioaffinity adsorbent and process for purification of immunoglobulin G also known as IgG, or antibody and fragments thereof such as Fab, Fc and F(ab)₂. The adsorbent comprises of (a) a solid support material also called here as base matrix, and (b) an interacting chemical group called a ligand as a part of the base matrix, or grafted on the base matrix, by any of the known activation chemistries, to give the desired characteristics such as the matrix hydrophobicity or hydrophilicity, group density and its spatial orientation so as to interact specifically with an IgG from its mixture with other proteins and biological substances.

Preferable structure of the ligand is,

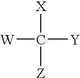

Where X, Y, Z and/or W is same or different and may be selected from the permutation and combinations of groups such as but not limited to H, amino, cyclohexyl alkenyl, alkenyloxy, alkoxy, alkoxyalkoxy, alkoxycarbonyl, alkyl, alkylcarbonyl, alkylcarbonyloxy, alkylsulfinyl, alkylsulfonyl, alkylthio, alkynyl, aryl, azido, arylalkoxy, arylalkyl, aryloxy, carboxy, cyano, formyl, halogen, haloalkyl, haloalkoxy, hydroxy, hydroxyalkyl, mercapto, nitro, sulfamyl, sulfo, sulfonate, hetrocyclic, benzene, riaphalene, indole, benzimidine, hydroxyphenyl preferably aryl and more preferably aryl having heteroatom such as nitrogen, sulfur, oxygen in the ring.

In the preferred embodiment of present invention X, Y, Z and/or NV is substituted or non-substituted, monocyclic or bicyclic system with or without heteroatom such as but not limited to nitrogen, sulphur, oxygen inside or etc. The term “mono- or bicyclic” is intended to mean that the core part of the moiety in consisting of one ring or two fused rings, e g as in benzene and indole respectively, and, also ligands comprising two separate rings such as in biphenyl.

In the preferred embodiment of present invention X, Y, Z and for W is substituted or non-substituted aliphatic or aromatic system with or without heteroatom such as but not limited to nitrogen, sulphur, oxygen etc.

In one embodiment of W, aryl group is an “indole group”, X is “amine”, Y is “carboxyl” and Z is “H” such as derivative of propanoic acid like in tryptophan.

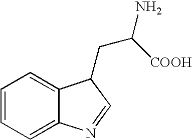

### Tryptophan: IUPAC name: 2-amino-3-(1H-indol-3yl)-propionic acid

In another embodiment of W, aryl group is “hydroxyphenyl” X is “amine”, Y is “carboxyl” and Z is “H” such as derivative of propanoic acid like in tyrosine

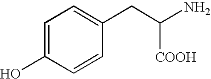

### Tyrosine: IUPAC name: 2-amino-3-(4-hydroxyphenyl)-propionic acid

In yet another embodiment of W, aryl group is “benzyl” X is “amine”, Y is “carboxyl” and Z is “H” such as derivative of propanoic acid like in phenylalanine

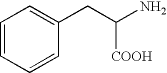

### Phenylalanine: IUPAC name: 2-amino-3-phenyl(1H-indol-3-yl)-propionic acid

From above discussed embodiment, specificity of binding of immunoglobulin to said adsorbents with above non limiting ligands grafted on solid support is in the order of tryptophan>tyrosine and phenylalanine. The ligand can he from the group of natural or synthetic and aliphatic or aromatic hydrophobic amino acids in D form or L form such as alanine, valine, leucine, isoleucine, proline, phenylalanine, tryptophan and tyrosine.

One skilled in art may readily use the same hydrophobic amino acid, or any permutation and combination of different hydrophobic amino acids. The solid support, or solid carrier, or the base matrix may be natural or synthetic and organic or inorganic material known applicable in solid phase separation of proteins and other biomolecules. Synthetic base matrix may comprise of hydrophilic polymer of methacrylate or acrylate species, or any of its derivatives such as polymethacrylate, polyacrylate, polymethylmethacrylate, polyhydroxyethylmethacrylate and/or polyglycidyl methacrylate. Natural or synthetic polysaccharides such as agar-agar and agarose, celluloses, cellulose ethers such as hydroxypropyl cellulose, carboxymethyl cellulose, starches, gums such as guar gum, and gum arabic, gum ghatti, gum tragacanth, locust bean gum, xanthan gum, pectins, mucins, dextrans, chitins, chitosans, alginates, carrageenans, heparins, gelatins. The base matrix can also be inorganic materials such as silicious materials such as silicon dioxide including amorphous silica and quartz; silicas; metal silicates, controlled pore glasses and ceramics; metal oxides and sulfides, or combinations of these natural or synthetic and organic or inorganic materials. Further, the base matrix may comprise polymer of polystyrene and divinyl benzene.

Furthermore, the solid phase matrix or support, as such, or as a result of suitable functionalization, may be in the form of (a) irregular particles or spherical beads, (b) membrane sheets, or (c) molded surfaces or sticks. The solid base matrix can made prepared from above mentioned polymers or commercially available media comprising above mentioned polymers or co-polymers can use as base matrix to graft or couple or attach the said pseudobioaffinity ligand. Some examples of commercially available base matrix for the preparation of said pseudobioaffinity adsorbent such as but not limited to Sepabeads (for example, Sepabeads FP-EP, Sepabeads EB-EP, Sepabeads FP-TIA) from Resindion SRL. Italy; BioRex, BioGel, Macroprep. UNOSphere from BioRad, USA; Agar or agarose beads such as sepharose and superose, sephacryl, superdex from GE healthcare, Sweden; Cellulose heads such as Celbeads from UICT, India; POROS media from Perseptive Biosystems. USA; cellulose membranes from Hydro Air Research, Italy can be used.

In a particular embodiment of the present invention, the solid support may be same or different. If different solid support is used, the difference lies in particle size, particle shape, pore size, pore volume, pore structure, group density, group orientation, hydrophobicity and hydrophilicity. In the preferred embodiment support matrix is in the form of irregular or spherical particles with sizes in the range of 5 μm to 2000 μm, preferably in the range of 20 μm to 1000 μm. Other properties of the matrices used in process of the present invention are surface area at least 50 m²/g, and pore diameter of at least 50 Å.

The ligand can be a part of base matrix, or may be attached covalently to solid support before or after preparation of matrix through various reactive groups such as but not limited to tosylates, tresylates, halides, epoxy group, cyanate esters, N-hydroxy succinimide esters, carbonyl diimidazole, aldehydes and like, on the solid support. The reactive groups can be introduced on the solid support by activating agents such as but not limited to epichlorohydrin, epibromohydrin, dibromo and dichloropropanol, bis-epoxides such as butanedioldiglycidylether, divinyl sulfone, tosyl chloride, tresyl chloride, and ethyleneglycol. Further, grafting of ligand can be done by using ionic or co-ordinate binds.

In an embodiment of the present invention the ligand is grafted or coupled to the base matrix with or without base matrix spacer arm. If a spacer arm is used, it consists of straight or branched chain of 1 to 10 carbon atoms and may or may not have branches comprising but not limited to amino, hydroxyl, carboxyl, sulfoxy, sufonyl, formyl, cyano, nitro etc.

In another embodiment of present invention the ligand density on said adsorbent is in the range of 1 to 500 μmol/ml, is preferably range of 10 to 350 μmol/ml and more preferable in the range of 10 to 200 μmol/ml.

In the context of present invention, the term “Immunoglobulin” refers to immunoglobulin G, IgG, antibody such as polyclonal or monoclonal, and fragments thereof such as Fab, Fc and F(ab)₂.

In the present disclosure the singular from “a”, “an” and “the”, includes plural reference also unless the context clearly deflect otherwise. Thus for example “The immunoglobulin containing solution” is reference to one or more types of solution containing immunoglobulin and equivalents thereof know to those skilled in art.

In accordance with the present invention the process comprises of equilibration, adsorption, washing, elution and regeneration, which can he carried out in batch or continuous mode. Batch operation can be carried out simple stirred tank like CSTR, packed bed, expanded bed mode, fluidized bed and/or moving bed. Further, the process of the present invents using said adsorbent cab be performed using annular, radial, centrifugal, or membrane chromatography.

In the present invention membranes can also be used as support matrix where the interacting groups and/or ligand is distributed on the surface of membrane and such system is used as membrane chromatography. The membranes used can be porous or nonporous and in the form of module such as but not limited to hollow fiber, flat sheet, spiral membrane. In the preferred embodiment of present invention the cross flow type of membranes were used to avoid concentration polarization effect.

In the embodiment of the present invention the said adsorbent with said ligand has a high degree of specificity and selectivity dictated by its binding constant, in the Langmuir adsorption isotherm of an order of 10⁻⁵ to 10⁻⁸, preferably of an order of 10⁻⁶ to 10⁻⁷ for immunoglobulins and is equivalent to Protein A for binding of immunoglobulins. This further dictates that use of said adsorbent bearing said ligand is responsible for getting high yield and purity of immunoglobulins.

The immunoglobulin containing solution of natural, recombinant, genetically engineered/modified, or transgenic sources or dried/semidried or lyophilized crude material obtained from said sources is directly contacted with the said adsorbent or is adjusted to desired pH and conductivity to promote the specific binding of IgG to the said pseudobioaffinity adsorbent at temperature of 4-30° C. most preferably at 15-30° C. The is in the range of 2.5 to 9.0. preferably 5.0 to 8.0; and salt content in terms of conductivity in range of 0.5 mS/cm to 50 mS/cm, preferably 1 mS/cm to 30 mS/cm. The adsorbent is pre-equilibrated with buffered aqueous solution having pH in the range of 2.5-9.0, preferably 5.0 to 8.0; and having a conductivity of 0.5 mS/cm to 50 mS/cm, preferably 1 mS/cm to 30 mS/cm. The immunoglobulin containing solution is then brought in contact with the adsorbent so that the IgG binds to the said pseudobioaffinity adsorbent. Said adsorbent is then washed with the equilibration solution to remove the unadsorbed or weakly adsorbed compounds. The selectively bound IgG is then eluted from the said adsorbent by contacting the said adsorbent with a buffer having a in the range of 2.5 to 9.0, preferably 5.5 to 8.0; and containing organic or inorganic acid salts so that the conductivity is in range of 20 mS/cm to 140 mS/cm, preferably 40 mS/cm to 120 mS/cm. The elution buffer can he required also to contain an additive such as but not limited to ethanol, ethylene glycol, glycerol, polyethylene glycol; sugars such as mono, di or polysaccharides etc. to enhance the recovery of the IgG in elution. Elution can he done in step gradient or linear pattern, wherein each step of gradient consists of at least one, two, three, four and/or five column volumes of elution buffer. The recovery of IgG obtained by the process of present invention is more than 80% with respect to crude source and more than 98% with respect to bound IgG. Purity of IgG recovered is at least 90%, at least 95% or as high as 100%. The said adsorbent may be then regenerated after the elution step to avoid contamination of the product and prevent the fouling of the adsorbent. The present pseudobioaffinity adsorbent can be treated with strong acidic solutions, alkaline solutions, and cleaning agents such as detergents, chaotropic salts and like which would otherwise be inappropriate with protein-based ligands. The cleaning solutions mostly used are sodium hydroxide solutions, potassium hydroxide solutions, solutions of peracids or hydrogen peroxide, organic solvents such as ethanol, guanidinium hydrochloride solutions, hypochlorite solutions etc. preferably 0.05-1.0M sodium hydroxide solutions. In the process of present invention the food grade acids, alkalies and salts are preferred. The mechanism of separation/purification of immunoglobulins from the immunoglobulin containing solution on said pseudobioaffinity is ionic, hydrophobic and/or mixed mode.

In an embodiment, of the present invention, wherein the solution containing the immunoglobulin fragments is contacted with the said adsorbent so as effect the selective binding of F(ab)2 fragment to the said adsorbent and Fc fragment remains in flow through or wash fractions, followed by washing of said adsorbent with washing solution, and desorbing bound F(ab)2 fragments using elution solution in high purity and yield.

The elution/desorbing solution may have a different pH, a different ionic strength, a different temperature and/or it may comprise detergents, chaotropes or other denaturing reagents. Combinations of one or more changes in these different conditions are also generally employed. The washing buffer while performing the present invention is not disturbing the binding of the immunoglobulins to the said adsorbent i.e. pH, salt concentration and other additives were adjusted so that only the unwanted impurities are removed either by simple substitution of the solution and impurities present in solution and around the adsorbent with the washing buffer or in combination herewith also releasing impurities bound to the adsorbent The releasing of impurities bound to the said adsorbent can be accomplished by changing pH and/or ionic strength or by adding a substance to the washing butler which interacts competitively with either the impurity, and thereby displacing the impurity from the adsorbent.

Regeneration procedure is typically performed regularly (i) to minimize the building up of impurities, (ii) to avoid fouling up the surface of the adsorbent, and (iii) to avoid contamination of the product with microorganisms proliferating and escaping from the adsorbent phase and the equipment used during the process. Typical solutions for these purposes would be, e.g., 0.1-1.0 M sodium hydroxide; solutions of peracids or hydrogen peroxide; denaturants such as guanidinium hydrochloride; solutions comprising active chlorine such as hypochlorite solutions, organic solvents such as ethanol; detergents etc. An especially preferred method for this purpose is to use 0.1-1.0 M sodium hydroxide due to the very high efficiency, low cost, ease of neutralization with hydrochloric acid and lack of waste problems.

In an embodiment where acids and/or bases were used, they can be selected from the group of organic or inorganic acids and/or bases such as but not limited to acids—acetic acid, citric acid, tartaric acid, hydrochloric acid, phosphoric acid, sulphuric acid, butyric acid and bases—sodium hydroxide, ammonium hydroxide, calcium hydroxide, and potassium hydroxide, amines such as trihydroxymethylaminomethane, dimethyl amine, alkylamines and any suitable combination of one or more thereof.

In an embodiment where salts were used, they can be selected from the group of salts of organic or inorganic acids and/or bases such as but not limited to salts of sodium ammonium, calcium, potassium as phosphates, carbonates, bicarbonates, acetates, citrates, tratarates and any suitable combination of one or more thereof.

In an embodiment where buffers were used, they can be selected from the group of salts of buffers from salts organic or inorganic acids and/or bases such as but not limited to acetate, phosphate, carbonate, 2-morpholino-ethane sulphonic acid (MES), glycine-HCl, Tris-HCl, and 4-(2-hydroxyethyl)-1-piperazine ethane sulphonic acid (HEPES) and any suitable combination of one or more thereof.

In an embodiment where the additives are used in equilibration, loading, washing, elution and/or regeneration solution, they can be selected from the group such as but not limited to ethanol, ethylene glycol, glycerol, polyethylene glycol; sugars such as mono, di or polysaccharides and any suitable combination of one or more thereof.

In the embodiment of present invention the term “gradient” includes stepwise, linear, convex and concave gradient effected in the composition/properties of the mobile phase used for selective desorption/elution of immunoglobulin/s. The term “column volumes” means the volume of mobile phase in which the final strength of eluting mobile phase is achieved.

Another embodiment of present invention, said pseudoaffinity adsorbent provides at least 25 to 120 mg/ml capacity to adsorb immunoglobulins from said sources.

Still another embodiment of present invention said pseudoaffinity adsorbent provides at least 25 mg/ml capacity to adsorb immunoglobulins from said sources.

Yet another embodiment of present invention said pseudoaffinity adsorbent provides at least 35 mg/ml capacity to adsorb immunoglobulins from said sources.

In another embodiment, said pseudoaffinity adsorbent provides at least 50 mg/ml capacity to adsorb immunoglobulins from said sources.

Another embodiment of present invention said pseudoaffinity adsorbent provides at least 75 mg/ml capacity to adsorb immunoglobulins from said sources.

Further another embodiment of present invention said pseudoaffinity adsorbent provides at least 90 mg/ml capacity to adsorb immunoglobulins from said sources.

Another embodiment of present invention said pseudoaffinity adsorbent provides at least 100 mg/ml capacity to adsorb immunoglobulins from said sources.

Another embodiment of present invention said pseudoaffinity adsorbent provides at least 120 mg/ml capacity to adsorb immunoglobulins from said sources.

In another embodiment of present invention said pseudoaffinity adsorbent was filled in the column and upward flow was given in order to fluidize the bed to 1.1 to 2.0 times of settled bed height. Immunoglobulin containing solution was then passed in upward direction through the column in expanded state so as to effect the adsorption of said immunoglobulin on said adsorbent without clogging the column. Adsorbent bed was then washed in expanded state followed by desorption of immunoglobulin in packed or expanded bed mode.

In an embodiment where desorption is carried out in expanded bed mode, the desorbing solution is passed in upward direction with a bed expansion of 1.01 to 2.0 times.

In yet another embodiment the said adsorbent is tilled into the column and feed is pumped into the column in upward or downward direction at a rate of 10-1200 cm/hr linear flow velocity preferably 75-800 cm/hr linear flow velocity.

In a preferred embodiment of the present invention the method includes the cycle steps such as (i) equilibration (optional step), (ii) contacting, (iii) washing (optional step), (iv) separation, (v) elution, and (vi) regeneration, where cycle of steps (i)-(v) are repeated one or several times before regeneration, and the solid phase matrix is reused after regeneration.

In an embodiment of present invention, said pseudobioaffinity adsorbent provides at least 80% to 100% recovery and purity of immunoglobulins from said sources.

In another embodiment said pseudobioaffinity adsorbent provides at least 80% recovery and purity of immunoglobulins from said sources.

Still another embodiment of present invention said pseudobioaffinity adsorbent provides at least 90% recovery and purity of immunoglobulins from said sources.

Yet another embodiment of present invention said pseudobioaffinity adsorbent provides at least 95% recovery and purity of immunoglobulins from said sources.

Further another embodiment of present invention said pseudobioaffinity adsorbent provides at least 100% recovery and purity of immunoglobulins from said sources

Immunoglobulins purified/separated by the said process can be further subjected to viral inactivation step, and can be used in the development of known immunoglobulin formulations. In simplest case the immunoglobulin may be used directly without any downstream treatment, but in many instances some sort of procedure would be preferred e.g. ultra-filtration, freeze-drying or precipitation (e.g. salting out). The immunoglobulin solution can be purified further in a processing step of optional character.

The present invention has various advantages over the known adsorbents and processes for purification of immunoglobulin such as:
1) Cost effective pseudoaffinity adsorbent as compared to many commercially available adsorbents such as Protein A. Protein G or Protein L based adsorbents, for purification of immunoglobulin/s.
2) Said adsorbent can be regenerated using harsh CIP/SIP protocols and reused
3) Use of a non toxic ligand and no leakage of ligand in the product
4) Can be operated effectively at high flow rates and at different temperatures
5) Immunoglobulin of more than 90% purity, and >80% recovery is obtained in a single column step without the use of any pretreatment like precipitation
6) Immunoglobulin can be purified from any source sample containing immunoglobulin.
7) No aggregate formation during elution
8) Process can be operated using said pseudobioaffinity adsorbent in an expanded/fluidized bed mode for feedstocks containing immunoglobulin and particulate matter.
9) The said adsorbent of the present invention can be use for isolation of proteins and other biomolecules. Proteins for examples, proteases such as pro-enzymes, trypsins, chymotrypsins, subtilisin pepsin, plasminogen, papain, renin, thrombin, and elastase, lipases, glucosidases, xylanases, lectinases; albumins; proteins from fermentations broths; protein from milk and whey; proteins from blood, plasma, and serum; proteins from fish waste; proteins from slaughter house waste such as organ and tissue extracts, example alkaline phosphatase from bovine intestines, and proteins from vegetable extracts such as potato, tomato, coconut, e g horse radish peroxidase.

### Examples

The following examples are for illustrative purposes only and should in no way limit the scope of invention.

### Example 1

### Preparation of Pseudobioaffinity Adsorbent

100 ml of Sepabeads FP-EP, a commercial epoxy activated porous polymethacrylate based matrix, was suspended in an equal volume of 50 mM sodium carbonate, pH 9.5 buffer containing 0.5 mmol/l tryptophan. Sepabeads FP-EP was obtained from Resindion SRL, Italy and this adsorbent matrix is in the form of polymethacrylate based nearly spherical rigid porous beaded resins. The suspension was then stirred at 50° C. for 48 hours. The adsorbent was then washed extensively with 1.0M NaCl and water to remove the unreacted ligand. The adsorbent was then suspended in 100 ml of 1.0M ethanolamine pH 9.0 and stirred at room temperature for 6 hours. The adsorbent was finally washed extensively with 1.0M NaCl and water and stored at 4° C. in de-ionized water before use. All purification examples below have used the pseudobioaffinity adsorbent thus made.

### Example 2

### Purification of Human Polyclonal IgG from Human Plasma

Sample Preparation: 100 ml human plasma was first diluted 1:1 with 25 mM sodium phosphate buffer, pH 7.0. The diluted human plasma was then diafiltered with 25 mM sodium phosphate pH 7.0 to adjust the pH to 7.0 and conductivity to 2 mS/cm.

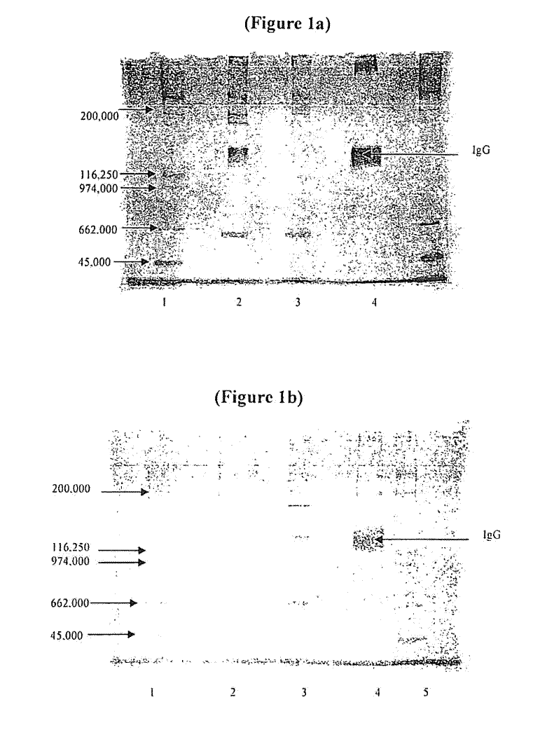

Experimental Setup: Chromatographic experiments were carried out using a 10 mm inner diameter and 100 mm long borosilicate glass column. 5 ml pseudobioaffinity adsorbent was packed in the column. The adsorbent was first equilibrated with 5 column volumes of 25 mM sodium phosphate buffer, pH 7.0 containing 10% PEG. To this equilibrated column 1 ml of above diafiltered human plasma was loaded at a linear velocity of 76 cm/hr. The adsorbent was then washed with 25 mM sodium phosphate buffer, pH 7.0 containing 10% PEG till the absorbance at 280 nm was 0.05. Elution was then performed by washing the adsorbent with 5 column volumes of 25 mM sodium phosphate, pH 7.0 containing 1.0 M NaCl and 20% polyethylene glycol 600. The adsorbent was then washed with 0.5 M NaOH to remove other impurities and clean the matrix. Quantification of the human polyclonal IgG in the unbound and elution fractions was done by HiTrap Protein G column (from, GE healthcare) and SEC column BioSil-250 (from BioRad, USA). The dynamic capacity of the pseudobioaffinity adsorbent for human polyclonal IgG at linear velocity of 76 cm/hr was more than 40 mg/ml. The recovery of the polyclonal human IgG was 95%. The total protein content of the fractions was determined spectrophotometrically at 280 nm. The bioactivity of the human polyclonal IgG was determined by using ELISA. The ELISA results confirmed that eluted fractions retained their bioactivity. The purity of the fractions was determined by applying samples of at least 10 μg of protein to SDS-polyacrylamide (7.5% w/v) slab gel electrophoresis (SDS PAGE) under non-reducing conditions (FIG. 1a). Silver staining method was used for visualization of the protein bands. The SDS PAGE analysis showed that eluted fraction contained IgG (band at 150 kD) in a highly pure form (FIG. 1a). Moreover the unbound fraction did not show any band at 150 kD (FIG. 1a).

### Example 3

### Purification of Horse Polyclonal IgG from Horse Serum

Sample Preparation: Horse serum containing IgG against snake venom was purified on the adsorbent. 50 ml of horse serum was first diluted 1:1 with 25 mM sodium phosphate buffer, pH 6.5. The diluted horse serum was then diafiltered with 25 mM sodium phosphate pH 6.5 to adjust the to 6.5 and conductivity to 2 mS/cm.

Experimental Setup: Chromatographic experiments were carried out using a 25 mm inner diameter and 100 mm long borosilicate glass column. 30 ml pseudobioaffinity adsorbent was packed in the column. The adsorbent was first equilibrated with 5 column volumes of 25 mM sodium phosphate buffer, pH 6.5. To this equilibrated column 55 ml of above diafiltered horse serum was loaded at a linear velocity 50 cm/hr. The adsorbent was then washed with 25 mM sodium phosphate buffer, pH 6.5 till the absorbance @280 nm was 0.05. Elution was then performed by washing the adsorbent with 5 column volumes of 25 mM sodium phosphate, pH 6.5 containing 1.0 M NaCl and 30% polyethylene glycol 600. The adsorbent was then washed with 0.5 M NaOH to remove other impurities and clean the matrix. Quantification of the horse polyclonal IgG in the unbound and elution fractions was done by HiTrap Protein G column (from GE Healthcare). The dynamic capacity of the pseudobioaffinity adsorbent for horse polyclonal IgG at linear velocity of 50 cm/hr was 6 mg/ml. The recovery of horse polyclonal IgG was 81%. The total protein content of the fractions was determined spectrophotometrically at 280 nm. The purity of the fractions was determined by applying samples of at least 10 μg of protein to SDS-polyacrylamide (7.5% w/v) slab gel electrophoresis (SDS PAGE) under non-reducing conditions. Silver staining method was used for visualization of the protein bands. The SDS PAGE analysis showed that eluted fraction contained IgG (band at 150 kD) with trace amounts of high molecular weight impurities (FIG. 1b). Moreover the unbound fraction did not show any band at 150 kD (FIG. 1b). It can be concluded from the SDS PAGE analysis that the horse polyclonal IgG in the eluted fractions had a purity of more than 90%.

### Example 4

### Purification of Monoclonal Antibody from Cell Culture Supernatant

Sample Preparation: The cell culture supernatant contained an industrially developed monoclonal antibody in concentration as low as 35 μg/ml. The cell culture supernatant was diafiltered with 25 mM Morpholinoethanesulfonic acid (MES) buffer, 6.5 to adjust the pH to 6.5 and conductivity to 3 mS/cm.

Experimental setup: Chromatographic experiments were carried out using a 10 mm inner diameter and 100 mm long borosilicate glass column. 5 ml pseudobioaffinity adsorbent was packed in the column. The adsorbent was first equilibrated with 5 column volumes of 25 mM Morpholinoethanesulfonic acid (MES) buffer, pH 6.5. To this equilibrated column 20 ml of above diafiltered cell culture supernatant was loaded at a linear velocity 50 cm/hr. The adsorbent was then washed with 25 mM Morpholinoethanesulfonic acid (MES) buffer, pH 6.5 till the absorbance @280 nm was 0.05. Elution was then performed by washing the adsorbent with 5 column volumes of 25 mM Morpholinoethanesul ionic acid (MES) buffer. pH 6.5 containing 1.0 M NaCl and 20% polyethylene glycol 600. The adsorbent was then washed with 0.5 M NaOH to remove other impurities and clean the matrix. Quantification of the monoclonal antibody was done by HiTrap Protein G column (from GE Healthcare). The recovery of monoclonal antibody was 96%. Purity of the monoclonal antibody in the elution fractions was determined by SDS PAGE analysis on 12% (w/v) polyacrylamide gel under reducing conditions. The gel was stained using silver staining procedure. This SDS PAGE analysis has shown only the bands of monoclonal antibody. Purity of monoclonal antibody obtained was 97.85%.

### Example 5

### Isolation of F(ab)₂ Fragments from the Digested Plasma

Sample preparation: 50 ml human plasma was first diluted 1:1 with 25 mM sodium phosphate buffer, pH 7.0. The diluted human plasma was then diafiltered with 25 mM sodium phosphate pH 7.0 to adjust the pH to 7.0 and conductivity to 2 mS/cm. This diafiltered human plasma was then digested with pepsin at 37° C., pH3.5 for 4 hr to get F(ab)₂ fragments.

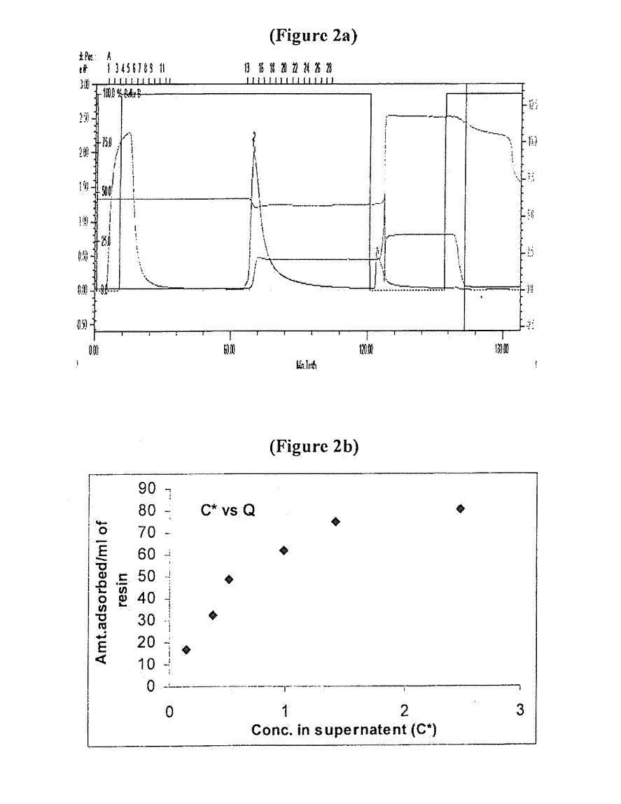

Experimental Setup: Chromatographic experiments were carried out using a 10 mm inner diameter and 100 mm long borosilicate glass column. 5.0 ml pseudobioaffinity adsorbent was packed in the column. The adsorbent was first equilibrated with 5 column volumes of 25 mM sodium phosphate buffer, pH 7.0 containing 10% PEG. To this equilibrated column 1 ml of above digested human plasma was loaded at a linear velocity of 76 cm/hr. The adsorbent was then washed with 25 mM sodium phosphate buffer, pH 7.0 containing 10% PEG till the absorbance @280 nm was 0.05. Elution was then performed by washing the adsorbent with 5 column volumes of 25 mM sodium phosphate, 7.0 containing 1.0 M NaCl and 20% polyethylene glycol 600. The adsorbent was then washed with 0.5 M NaOH to remove other impurities and clean the adsorbent (FIG. 2a). The purity of the fractions was determined by applying samples of at least 10 μg of protein to SDS-polyacrylamide (7.5% w/v) slab gel electrophoresis (SDS PAGE) under non-reducing conditions. Silver staining method was used for visualization of the protein bands. The SDS PAGE analysis showed that eluted fraction contained F(ab)₂ (band at 100 kD) in a highly pure form. Moreover the unbound fraction did not show any band at 100 kD. The purity of F(ab)₂ in the eluted fractions 96.89%.

### Example 6

### Adsorption Isotherm of Human Polyclonal Immunoglobulin G of Pseudobioaffinity Adsorbent

The adsorption isotherm provides values of the binding capacity and affinity of the pseudobioaffinity adsorbent to IgG. 0.5 ml of said pseudoaffinity adsorbent was contacted with 5 ml of immunoglobulin G solution of various concentrations. The adsorption data was fit directly to a Langmuir model and the results are shown in FIG. 2b. Values obtained for Qₘₐₓ and Kd are 100 mg/ml and 6.34×10⁻⁶M respectively. This indicates that the said ligand has high specificity and selectivity, equivalent to Protein A.

### Example 7

### Leakage Test for Said Ligand from Said Pseudobioaffinity Adsorbent

Said pseudobioaffinity adsorbent, 5 ml was kept in contact of 50 ml of each DM water; equilibration, washing, loading, elution and regenerating solution and in 70% ethanol on rocking platform for 7 days and leakage of ligand into solution was analyzed by using Zorbax SB-C18, 250×4.6 mm, 5μ (Agilant technologies), with detection at 205 nm and 280 nm using photodiode array detector and at flow rate of 1 ml/min. Each time 20 μl of supernatant sample was injected and chromatogram was monitored for 60 min. No any peak corresponding to standard tryptophan was observed. Also not as single unknown peak was observed with respect to blank preparations for each solution tested after 12 hrs, 24 hrs and after 7 days for all solutions.

### Example 8

### HPLC Assay Purity of Immunoglobulins Recovered by Using the Process and Said Adsorbent of the Present Invention

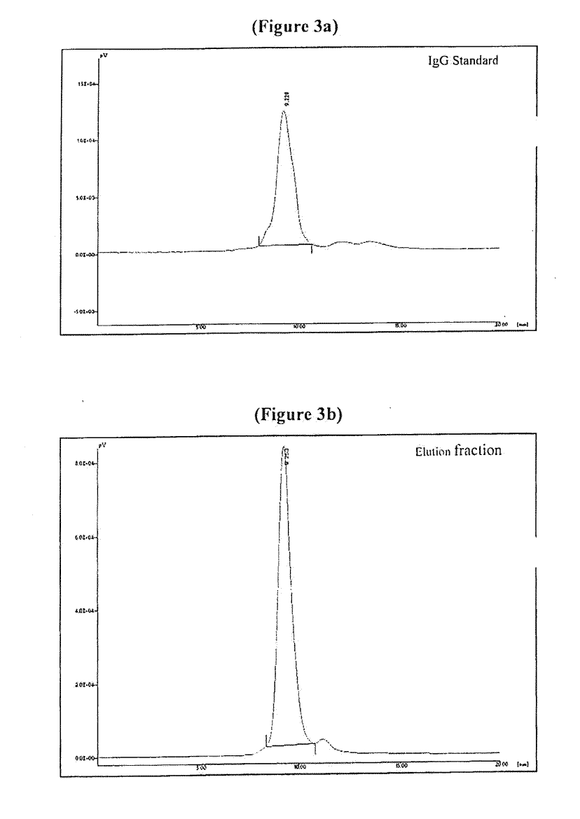

Human Immunoglobulin, IgG was purified using the process described examples 2. The assay purity of the recovered IgG was determined using SEC column BioSil-250 (from BioRad, USA). FIG. 3a and FIG. 3b shows the HPLC chromatograms of human standard IgG and purified human IgG respectively. Assay purity of purified human IgG was then calculating the area of standard and purified sample. It was found that human IgG recovered has assay purity of 99.23%.

1.-13. (canceled)

14. A process for immunoglobulin purification/separation using pseudobioaffinity adsorbent comprising:
a. providing a rigid, porous pseudobioaffinity adsorbent comprising an amalgamation of a hydrophilic polymer base/support and attached/immobilized affinity ligand/s including (i) aliphatic and/or aromatic propanoic acid derivative/s and/or (ii) aliphatic or aromatic hydrophobic amino acids, or (iii) any suitable combinations thereof with average ligand density between 0.1 to 0.2 mol/lit (i.e. 100 to 200 μmol/ml) with or without a spacer arm and leading to ionic, hydrophobic and/or mixed mode interaction/s between the said pseudobioaffinity adsorbent and an immunoglobulin;
b. contacting the said adsorbent with an immunoglobulin containing solution directly, or after adjusting the pH and conductivity of the immunoglobulin containing solution to adsorb the immunoglobulins on the said adsorbent with high specificity and high selectivity with a high immunoglobulin adsorption capacity between 25 to 120 mg immunoglobulins/ml (i.e. 33 to 160 g/kg) of adsorbent;
c. optionally washing the said adsorbent with a washing solution;
d. contacting the said adsorbent with desorbing solution in order to elute the bound immunoglobulin and fragments thereof with recovery and purity of 80% to 100% and 90 to 100% respectively; and
e. optionally, regenerating and equilibrating the said adsorbent for reuse.

15. A pseudobioaffinity adsorbent comprising an amalgamation of a rigid porous hydrophilic polymer base/support material of methacrylate or acrylate species, or any of its derivatives and attached/immobilized affinity ligand/s which is selected from a group consisting of (i) aliphatic and/or aromatic propanoic acid derivative/s and/or (ii) aliphatic or aromatic hydrophobic amino acids, and (iii) any suitable combinations thereof with average ligand density between 0.1 to 0.2 mol/lit (i.e. 100 to 200 μmol/ml) with or without a spacer arm; said adsorbent having high specificity and high selectivity with a high immunoglobulin adsorption capacity between 25 to 120 mg immunoglobulins/ml (i.e. 33 to 160 g/kg) of adsorbent.

16. A pseudobioaffinity adsorbent of claim 15, wherein the rigid porous hydrophilic polymer support material comprises synthetic hydrophilic polymer of acrylate or methacrylate species, or compounds, or any of their derivatives selected from a group consisting of polymethacrylate, polyacrylate, polymethylmethacrylate, polyhydroxyethyl-methacrylate or polyglycidyl methacrylate, or their combinations within themselves, or with other natural or synthetic copolymers.

17. A pseudobioaffinity adsorbent of claim 15, wherein the ligands support material is selected from the group consisting of a methacrylate polymer, and a co-polymer of methacrylate and acrylic acid.

18. A pseudobioaffinity adsorbent of claim 15, wherein ligand is selected from the group consisting of propanoic acid derivatives, natural amino acids, synthetic amino acids, aliphatic amino acids, aromatic hydrophobic amino acids in D form or L form, alanine, valine, leucine, isoleucine, proline, phenylalanine, tryptophan and tyrosine coupled to the rigid support.

19. A pseudobioaffinity adsorbent of claim 15, wherein ligand is selected from the group consisting of a derivative of propanoic acid, an aromatic hydrophobic amino acid and tryptophan, coupled to the rigid and porous support.

20. A pseudobioaffinity adsorbent of claim 15, wherein the support material is in the form of spherical beads, irregular particles, membrane sheets, molded surfaces or sticks.

21. A pseudobioaffinity adsorbent of claim 15, wherein the support material is in the form of spherical beads of size in the range of 5 μm to 2000 μm.

22. A pseudobioaffinity adsorbent of claim 15, wherein the support material is in the form of spherical beads of size in the range of 20 μm to 1000 μm.

23. A pseudobioaffinity adsorbent of claim 15, wherein the ligand is attached to the support matrix, with or without a spacer arm, or it is attached to the monomeric and oligomeric constituents of the matrix prior to polymerization to form an adsorbent matrix.

24. A process of separation/purification of immunoglobulin/s, said process comprising:
a) packing pre-equilibrated pseudobioaffinity adsorbent in a chromatographic column;
b) contacting immunoglobulin containing solutions with said pre-equilibrated pseudobioaffinity adsorbent for selective binding of immunoglobulin to the pseudobioaffinity adsorbent under the conditions of pH in the range of 2.5 to 9.0, and salt content in terms of conductivity in the range 0.5 mS/cm to 50 mS/cm, where contaminating proteins remain in the unadsorbed fraction;
c) washing the pseudobioaffinity adsorbent with a buffer, having the same or near properties of pH and salt strength as in step (b) above, to remove unadsorbed, or weakly adsorbed, compounds;
d) eluting the bound immunoglobulin in 80% to 100% recovery and 90% to 100% purity by washing the pseudobioaffinity adsorbent with desorbing buffer having pH in the range of 2.5 to 9.0, and containing ionic salts so that conductivity is in the range of 20 mS/cm to 140 mS/cm and also containing additives selected from a group consisting of alcohol, polyols, ethanol, sugars, polyethylene glycol, ethylene glycol and glycerol; and
e) regenerating and re-equilibrating the pseudobioaffinity adsorbent for reuse.

25. The process according to claim 24, wherein the pseudobioaffinity adsorbent is contacted with the immunoglobulin containing solution, washing solution, eluting solution, regenerating and equilibrating solution in a mode selected from the group consisting of batch mode, stirred batch mode, packed bed mode, expanded bed, fluidized bed mode and moving bed.

26. The process according to claim 24, wherein the buffer is selected from the group consisting of acetate, phosphate, MES, carbonate, glycine-HCl, Tris-HCl and HEPES.

27. The process according to claim 24, wherein the additives added to the buffer are selected from the group consisting of ethanol, ethylene glycol, glycerol, polyethylene glycol and sugars.
